(19) United States
(12) Patent Application Publication
(10) Pub. No.: US 2005/0028825 A1
(43) Pub. Date: Feb. 10, 2005

(54) Condom cover all

(76) Inventor: Leonard McCoy, Ann Arbor, MI (US)

Correspondence Address: Mr. Leonard McCoy, 1860 Northbrook Drive, Ann Arbor, MI, 48103, US

(21) Appl. No.: 10/619,670

(22) Filed: Jul. 15, 2003

(51) Int. Cl. A61F 6/04

(52) U.S. Cl. 128/844

(57) ABSTRACT

Applicant's invention is a condom for use on a natural or artificial penis. The condom comprises a single body with two different sections. The first section is an elongated generally tubular section and has a closed end. The second section extends seamlessly from the first section has an open end and is shaped to fit the scrotum of a user, without any excess condom membrane. A one-quarter-inch (¼″) thick resilient means is adjacent the open end of the second section. The resilient means expands to fit over the penis and scrotum and to size itself to fit securely and comfortably between the scrotum and the torso of the user. A package for multiple condoms has a slanted front with tear tab for easy access.

### REFERENCE TO RELATED APPLICATION

This application is based on U.S. patent application Ser. No. 10/619,670, filed Jul. 15, 2003

### BACKGROUND OF THE INVENTION

The field of the invention pertains to condoms and, in particular, to a condom that provides greater coverage to the user.

Historically, condoms have been used on a male's penis for the prevention of pregnancy by limiting the travel of the ejaculate and sperm from a male to a female. Sexually transmitted diseases such as syphilis, gonorrhea, etc. could be limited by the use of a condom during sexual intercourse. More recently, limiting the transfer of Human Immune Virus (HIV) that can lead to the fatal disease of Auto Immune Deficiency (AIDs) has become of critical importance. Protection from viral and bacterial diseases requires elimination of transfer of bodily fluids between a condom user and the sexual partner.

Two major problems with condoms as they are currently known is that there can be slippage during use (condom comes off or rolls down to the point that it allows passage of bodily fluids between the sexual partners) and spillage as the condom is being removed from the penis after use. There have been attempts to address these problems through the use of harnesses that secure the condom to the body (U.S. Pat. No. 6,123,079; U.S. Pat. No. 6,102,043; U.S. Pat. No. 5,070,890), two or more piece condoms (U.S. Pat. No. 6,478,027; U.S. Pat. No. 6,209,543; U.S. Pat. No. 5,718,236; U.S. Pat. No. 5,314,447; U.S. Pat. No. 5,070,890), use of preformed scrotal cups to provide complete coverage of the genital area (U.S. Pat. No. 6,209,543; U.S. Pat. No. 5,718,236; U.S. Pat. No. 5,318,042; U.S. Pat. No. 5,314,447; U.S. Pat. No. 5,070,890).

All the patents cited above that use a harness to secure the condom to the user have the same disadvantage of being difficult to apply and use, in that harnesses require that the user stop the activity being engaged in to tie or wrap the harness and the harness may come undone or break. Applicant's new condom is a significant improvement on these designs in that the user does not have to learn anything new to use this condom and it is more secure

All the patents cited above for condoms that are two or more pieces suffer from difficulty of use as well as inconvenience. If the user in haste does not properly adjust straps, ties or additional pieces, the protection that the overall condom provides is lost. Applicant's new condom does not require these adjustments to provide the benefits of an overall condom.

All the condoms with preformed rigid scrotal cups cited above have the disadvantage of being one size (which may or may not fit), the preformed scrotal cups that are not rigid have excess condom membrane material in scrotal area (which may chafe, tear or crack during use or application) and the rigid preformed cup is difficult to position. Applicant's new condom has the advantage of conforming exactly to the size and shape of the scrotum without excess material.

As to U.S. Pat. No. 5,318,042, which is also a one piece condom with a bulbous type scrotal sac, the disadvantage of rigid construction and limited access to the scrotal sac is not practical and contains no securing or restraining device to keep the condom in place on the user.

A condom is the most readily available and effective means of limiting transmission of bodily fluids during intercourse. With a view toward overcoming some of the problems of condoms as stated above, applicant has developed the following full coverage condom.

The most readily available and best selling condoms (traditional) only cover the penis of the male. However, bodily fluids can leak from around an open end of traditional condoms because of slippage or spillage when the penis is being removed from the sexual partner. Patents for condoms of varying designs (as cited above) have been granted to address these problems. The male can hold the top of a known condom around the open end but sometimes the condom can slip off the penis before the penis is fully removed from the area of the vagina of a female or other location. Other condoms designed to overcome these problems employ the cover all design but accomplishes this with one or more of the following methods; two or more pieces, drawstrings to secure the condom to the user, and the securing devices in front of the scrotum.

Thus, is needed a more practical and safer condom.

### SUMMARY OF THE INVENTION

Applicant's new condom is a condom cover all or a full coverage condom. The new condom is a single molded piece with two distinct sections. The first section has a closed end for holding seminal fluid near the tip of a penis. The first section forms an elongated generally tubular section designed to fit a natural penis of a user or an artificial penile facsimile. The first section has a traditional wall thickness. The first section seamlessly extends into the second section.

The second section is designed to fit around and support the scrotum of the user. The second section extends from the first elongated generally tubular section with the second section designed to fit the scrotum of a user. The second section expands to fit any size scrotum without any loose material. The wall thickness of the second section can be the same wall thickness of the first section, but may be thicker or thinner.

The second section has an open end with a quarter-inch (¼″) thick elastic or resilient means (gripper) adjacent to the open end adapted to hold the condom securely to a user. The open end with the quarter-inch (¼″) elastic ring fits between the scrotum and the torso. The elastic ring can be expanded sufficiently to be placed over the penis and the scrotum and return to a non-expanded position that is sufficient to comfortably grip the condom to the user or to the penile facsimile. The gripper forms a round shape to the open end or a square shape or another shape. The condom can be sized in different sizes as needed for different users. Thus, Applicant's condom is applied to the penis much as the traditional condom is without the additional securing devices.

The condom can be formed from condom materials such as latex rubber, animal skin, or synthetic membrane. Ribs can be added to a section or sections of the condom to provide pleasure for the partner. The ribs can take a variety of styles.

In the wrapping or packaging for the individual condom, the two sections of the condom are not discernable. The second section is packaged in a collapsed state and expands when applied to fit the scrotum of a male. When the condom is removed from the package, a mark is displayed to indicate the top of the condom to aid in the proper application.

The individually wrapped condoms can be further packaged in a condom box that can hold multiple individually packaged condoms. It is desirable to use a slanted box for a package with a tear tab from the front slanted side extending part way across the topside of the box for easy access. Upon opening the tear tab of the packaging box, the multiple individually packaged condoms are displayed. The lowest condom package in the box is the nearest to the front of the box. The next higher condom package is positioned slightly farther into the slanted box as is the next higher condom package. The tear tab can be closed after a condom has been selected to protect the remaining condoms.

For a more complete understanding of the present invention, reference is made to the following detailed description when read in conjunction with the accompanying drawings wherein like reference characters refer to like elements throughout the several views.

### BRIEF DESCRIPTION OF THE DRAWINGS

FIG. 1 illustrates a view of the new condom according to the invention positioned on a user's body:

FIG. 2 illustrates a view of the new condom with resilient means forming a round open end;

FIG. 3 illustrates a partial view of the condom with resilient means forming a square open end;

FIG. 4 illustrates a perspective view of packaging for multiple individually wrapped condoms;

FIG. 5 illustrates a view of the new condom unrolled with the scrotum cup collapsed;

FIG. 6 illustrates a view of the new condom unrolled from the top and the scrotum cup partially expanded below;

FIG. 7 illustrates a side view of the new condom with the scrotum cup partially expanded;

FIG. 8 illustrates the initial placement of the new condom on the user's penis;

FIG. 9 illustrates the new condom unrolled to the user's scrotum;

FIG. 10 illustrates the new condom stretching over the user's scrotum and the scrotum cup expanding;

FIG. 11 illustrates the new condom fully unrolled, covering the scrotum, and secured in place by the one-quarter-inch (¼″) thick resilient means (gripper) end piece between the scrotum and the torso of the user; and

FIG. 12 illustrates the new condom as it is removed from the package with manufacture's mark of choice displayed to distinguish the top of the new condom from the scrotal side as an aid to the user to apply.

### DESCRIPTION OF THE PREFERRED EMBODIMENT

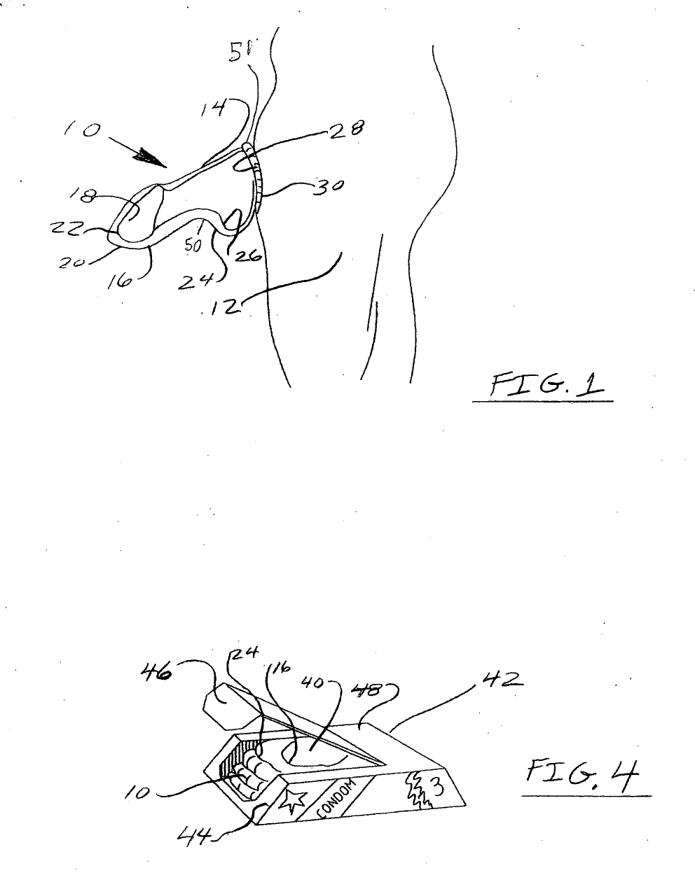

Illustrated in FIG. 1 new condom 10 is there shown worn by user 12. Condom 10 is comprised of two different sections in a single body 14 of condom 10. First section 16 fits onto penis 18 of user 12. First section 16 has closed end 20 that fits to tip 22 of penis 18. Second section 24 fits around scrotum 26 of user 12 leaving no excess condom membrane 50 around scrotum 26. Resilient means 28 near open end 30 holds condom 10 securely to male 12.

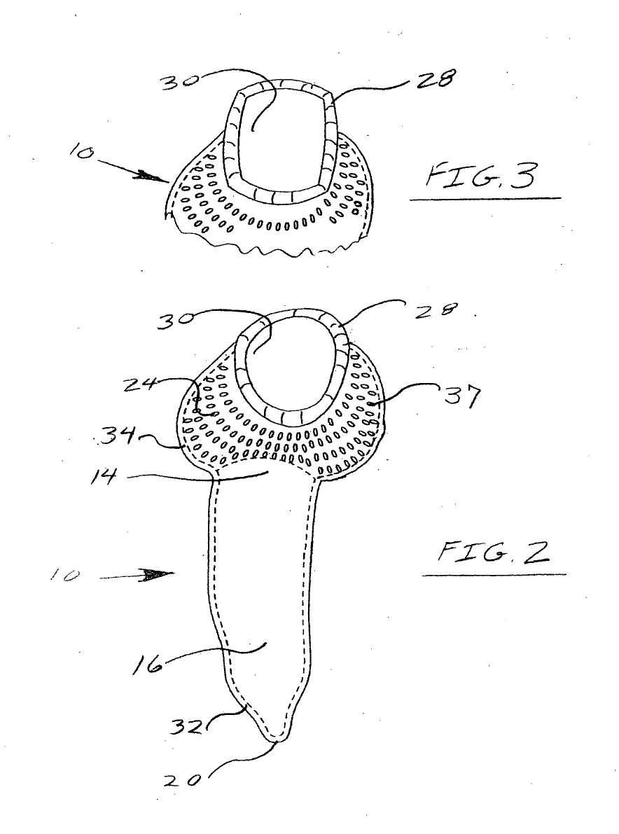

Now turning to FIG. 2 condom 10 is depicted. First section 16 of single body 14 extends into second section 24 of single unitary body 14 of condom 10. Wall thickness 32 of first section 16 can be more, less or the same as wall thickness 34 of second section 24. Resilient means 28 near open end 30 is shown with a round configuration in FIG. 2. Ribs 37 can be added to a section of the condom 10. Ribs 37 are here shown as added to second section 24 of single body 14.

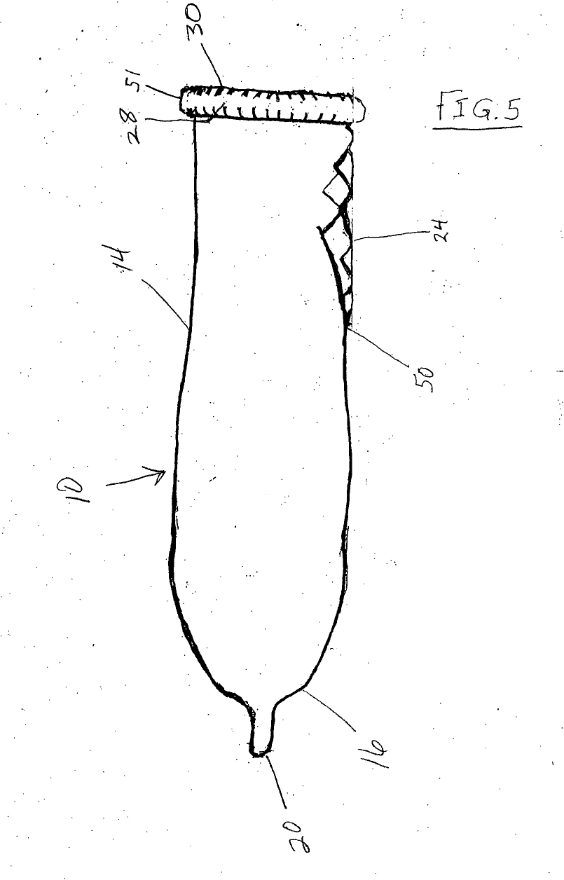

FIG. 3 shows resilient means 28 near open end 30 of condom 10 as forming a square configuration.

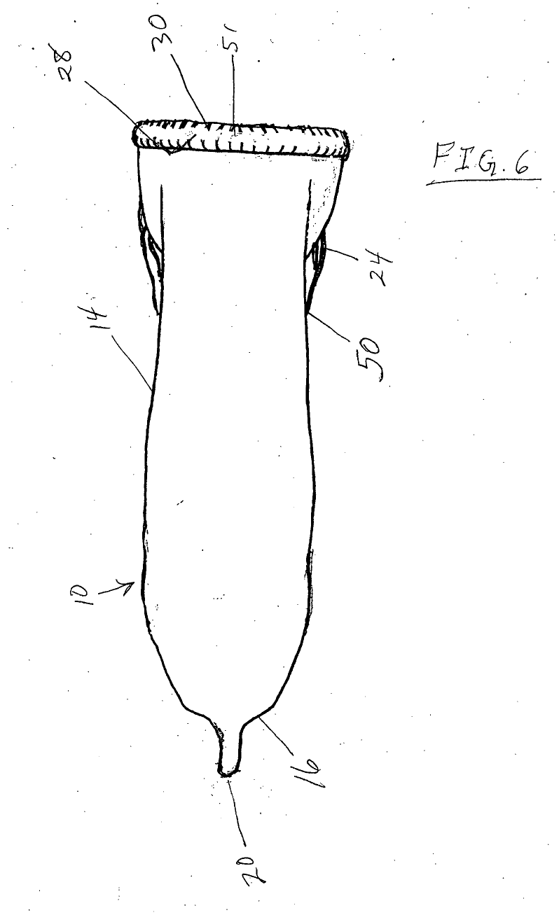

FIG. 4 shows individual packaged condoms 40 as packaged in a multiple condom package 42. The first section 16 and second section 24 of condom 10 are not discernible through individual condom packaging. Multiple condom package 42 for storing multiple individual condoms 10 has a slanted front 44 with tear tab 46 on slanted front and extending part way across the top 48 of package 42 for easy access. Condoms 10 in the package 42 are protected and less noticeable than when kept in a pocket.

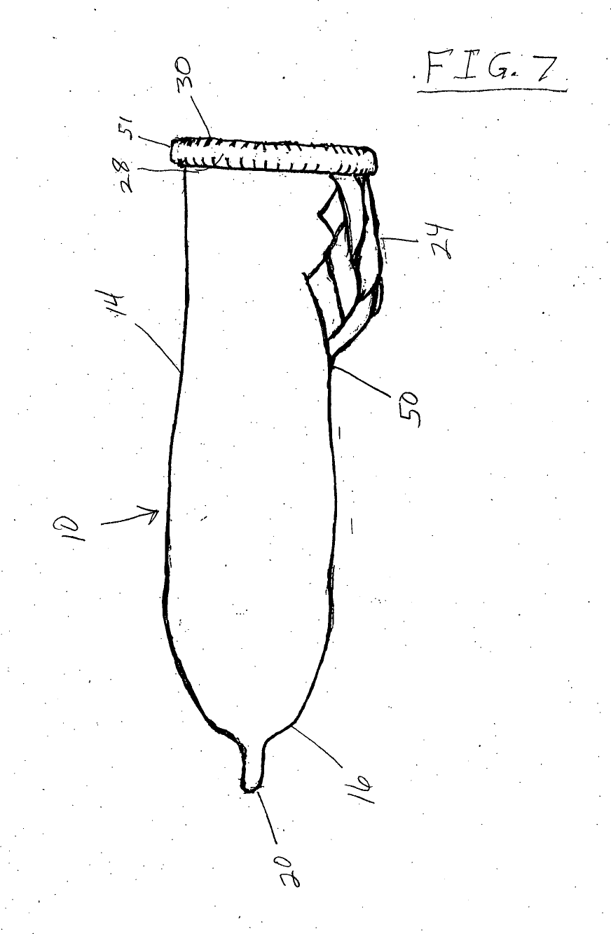

FIG. 5 shows unrolled condom 10 with second section 24 in its collapsed state for ease of packaging and application to user.

FIG. 6 shows top view of unrolled condom 10 with partially expanded second section 24.

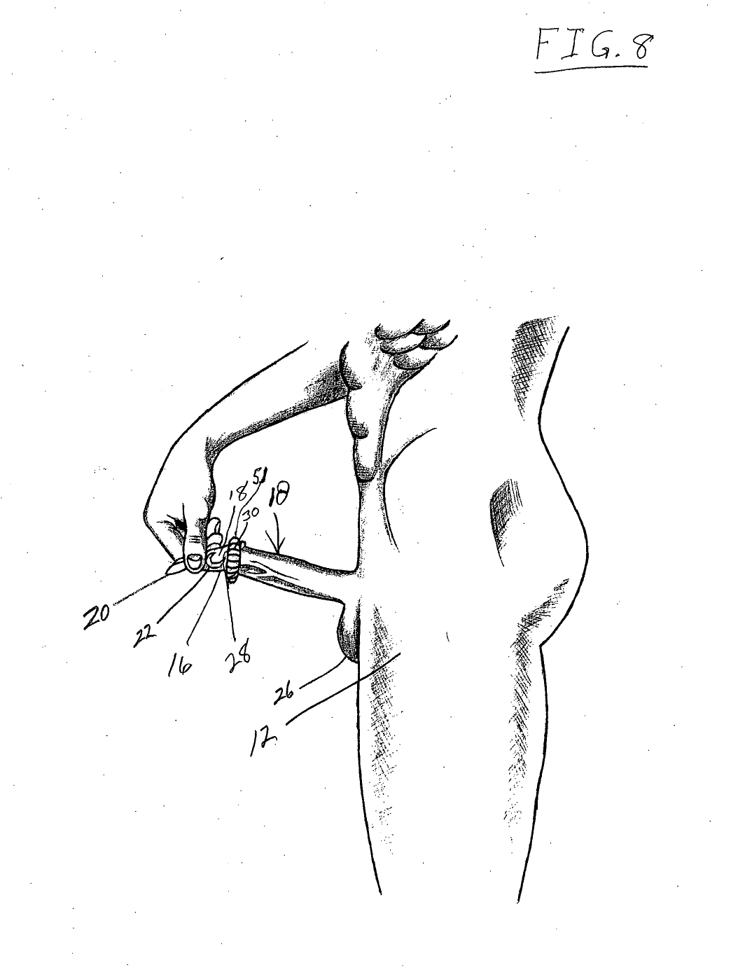

FIG. 7 shows side view of unrolled condom 10 with second section 24 partially expanded.

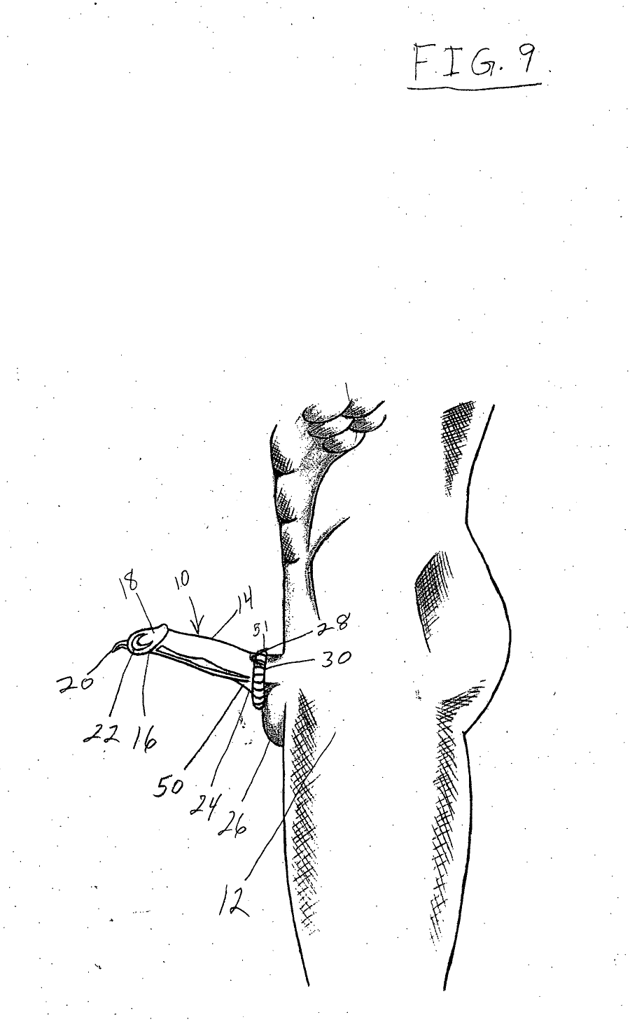

FIG. 8 shows user 12 applying condom 10 to penis 18 with manufacturer's mark 51 on top.

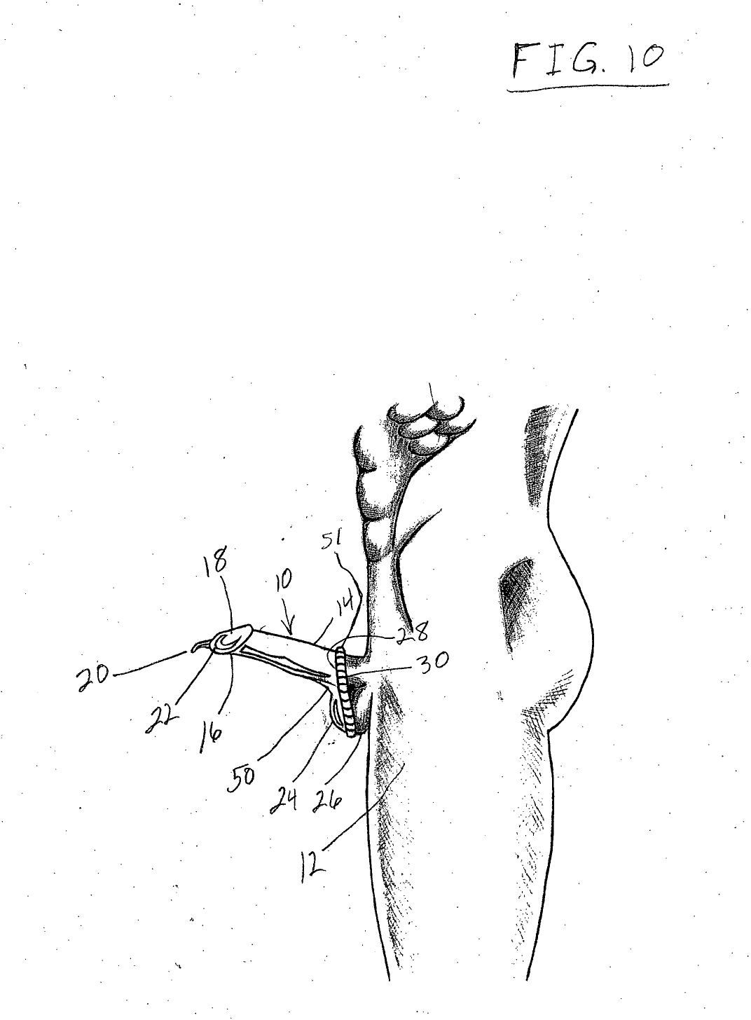

FIG. 9 shows condom 10 on penis 18 partially unrolled to scrotum 26 with resilient means 28 in front of scrotum.

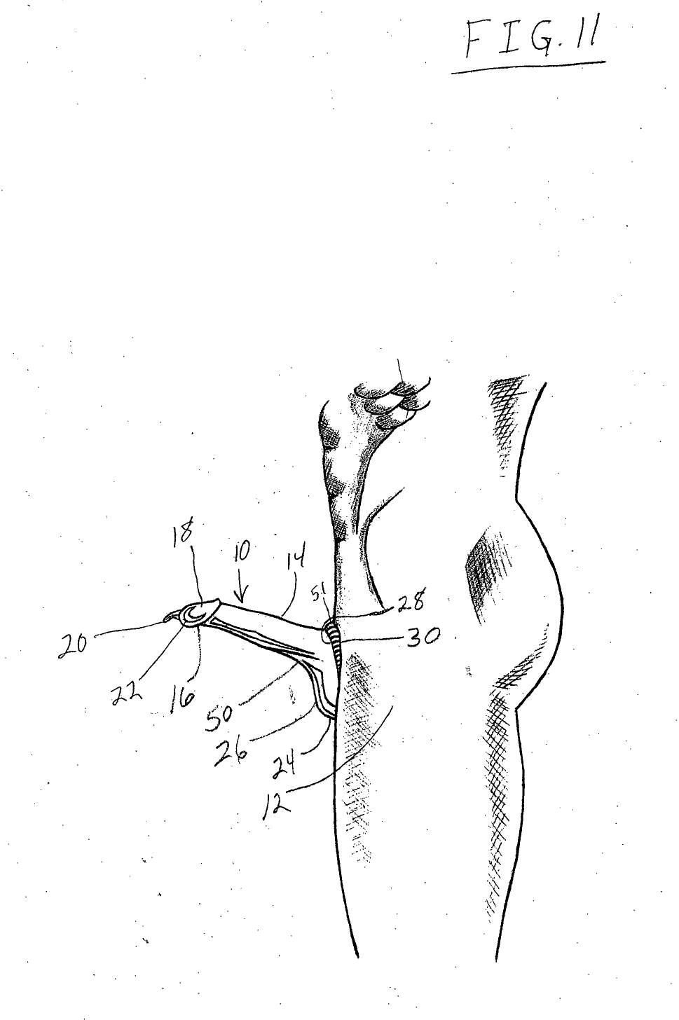

FIG. 10 shows condom 10 on penis 18 with second section 24 and resilient means 28 expanding around scrotum 26 with no excess condom membrane 50 in front of scrotum 26 and penis 18.

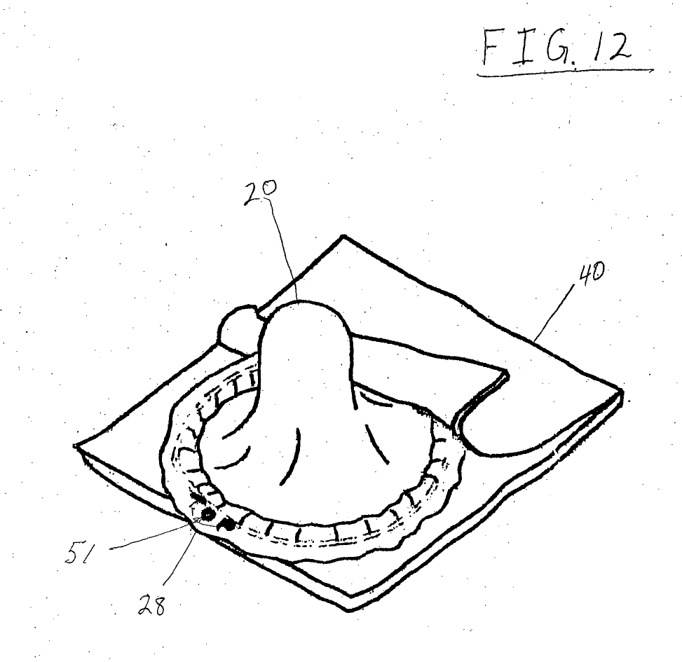

FIG. 11 shows condom 10 fully applied to penis 18 and scrotum 26 with resilient means 28 securing condom 10 to user 12 between scrotum 26 and torso of user 12, leaving no excess condom membrane 50 of second section 24 around scrotum 26.

FIG. 12 shows condom 10 being removed from individual package 40 with manufacture's mark 51 to indicate top from bottom of condom 10 to eliminate the prospect of applying second section 24 to the top of penis 18.

1. A male condom comprising:
a unitary body having a closed end and an open end, the unitary body having a first elongated generally tubular section adapted to fit a penis of a male, the first elongated generally tubular section having the closed end,
a second section extending from the first elongated generally tubular section, the second section being in a collapsed state that expands to conform to the size and shape of the scrotum of a male, the second section having the open end, and resilient means adjacent the open end adapted to hold the condom securely to a male, the resilient means of one quarter-inch (¼″) thickness adapted to fit between the scrotum and the torso of a male.

2. The condom of claim 1, wherein the unitary body comprises one of latex rubber, animal skin or a synthetic membrane.

3. The condom of claim 1, wherein the resilient means adjacent the open end allows expansion of the open end of second section over larger size of scrotum and then reduces and grips a smaller size between the scrotum and a torso of a male

4. The condom of claim 3 wherein the resilient means can be configured in one of a round or square shape.

5. The condom of claim 4 wherein the resilient means being adjustable and flexible.

6. The condom of claim 1 wherein the second section is in a collapsed state before being applied to the male penis and expands to exactly fit the scrotum of the male, without any excess or loose condom membrane.

7. The condom of claim 1, wherein the first elongated generally tubular section adapted to fit a penis of a male having a first wall thickness, and the second section extending from the first elongated generally tubular section, the second section being adapted to fit a scrotum of a male, the second section may be the same thickness, thinner, or thicker.

8. The condom of claim 7 wherein the second section is preformed with a larger area shaped for a male scrotum, the preformed area is collapsed and expands for an exact fit for a male scrotum.

9. The condom of claim 1 wherein the condom can be sized for a multiplicity of sizes for a variety of individual user.

10. The condom of claim 1 wherein the condom is packaged flat with the top of the condom marked to distinguish the top of the condom from the bottom (scrotum) side.

11. The condom of claim 1 further comprising packaging where at least a multiplicity of individually packaged condoms is provided.

12. Packaging for a condom of claim 11 wherein at least a multiplicity of individually packaged condoms are displayed with each packaged condom being placed above and back of a lower condom.

13. The condom of claim 12 where the packaging is further comprised of the ability to be opened and closed wherein at least a multiplicity of individually packaged condoms are available for selection.

14. A condom for use by a user for the prevention of pregnancy and the prevention of disease, the condom comprising:
a body having a closed end and an open end, the body having a first section adapted to fit a penis or a penile facsimile, the first section having the closed end,
the second section being in a collapsed state that expands to conform to the size and shape of the scrotum of a male or a genital facsimile, the second section having the open end, and
a resilient means (gripper) of one quarter-inch (¼″) thickness near the open end of the second section adapted to hold the condom securely to a user between the scrotum and the torso.

15. A condom for use in the prevention of pregnancy and the prevention of disease, the condom comprising:
a body having a closed end and an open end, the body having a first section adapted to fit a penis and a penile facsimile, the first section having the closed end,
the second section being in a collapsed state that expands to conform to the size and shape of the scrotum of a male and a genital facsimile, the second section having the open end, and
a resilient gripper means of one quarter-inch (¼″) thick near the open end of the body adapted to hold the condom securely to a user and adapted to fit between the scrotum and the torso of a user and or a genital facsimile.

16. The condom of claim 1 resilient means of the open end of the condom is one-quarter-inch (¼″) thick; this thickness makes the three (3) step application of the condom very easy for the user, in particular the positioning of the second section, expandable scrotum pouch, over the scrotum; this thickness provides enough material to build in enough tension to prevent transfer of bodily fluids, without breaking or splitting, slipping or coming off until it is removed by user; this thickness makes it possible to construct the resilient means to also function as a device to prolong an erection, when additional tension is built in. It also makes its removal very easy because of its size. One-quarter-inch is the optimal size for the resilient means, but could vary.

17. A method of use of the condom according to claim 1 comprised of the following three easy steps; (1) position the thumb and forefinger around the closed end of the condom, (2) unroll the first generally tubular section of the condom to the scrotum, (3) expand the open end resilient means over the scrotum, which causes the second section of the condom to expand to fit the scrotum (without any excess condom membrane between the penis and the scrotum or between the scrotum and the torso) and the resilient means sizes itself to the size of the penis between the scrotum and torso and holds the condom securely and comfortably in place.
